(19) United States
(12) Patent Application Publication
(10) Pub. No.: US 2023/0148879 A1
(43) Pub. Date: May 18, 2023

(54) COMPUTER-BASED PLATFORMS AND SYSTEMS CONFIGURED FOR CUFF-LESS BLOOD PRESSURE ESTIMATION FROM PHOTOPLETHYSMOGRAPHY VIA VISIBILITY GRAPH AND TRANSFER LEARNING AND METHODS OF USE THEREOF

(71) Applicant: Rutgers, The State University of New Jersey, New Brunswick, NJ (US)

(72) Inventors: Weinan Wang, New Brunswick, NJ (US); Laleh Najafizadeh, New Brunswick, NJ (US)

(21) Appl. No.: 17/980,367

(22) Filed: Nov. 3, 2022

Related U.S. Application Data
(60) Provisional application No. 63/275,189, filed on Nov. 3, 2021.

(51) Int. Cl. A61B 5/021; G06T 7/50; G06V 10/44; G06V 10/764

(52) U.S. Cl. CPC A61B 5/021; G06T 7/50; G06V 10/44; G06V 10/764; G06T 2207/20084

(57) ABSTRACT

A method includes receiving signal data from a sensor device; dynamically converting the signal data into a visibility point based on a time series vector associated with the signal data; generating an image to preserve the time series vector, wherein the a time series vector is a shape within the image; extracting a feature metric of a plurality of feature metrics from the image based on an analysis of a pre-trained machine learning algorithm; automatically determining, utilizing a transfer learning algorithm, a first position of a node in a plurality of nodes within the image based on a relationship between the feature metric and the time series vector associated with the time series data; predicting a second position of the node in the plurality of nodes based on the analysis of the pre-trained machine learning algorithm and the relation between the feature metric and the time series vector.

### CROSS-REFERENCE TO RELATED APPLICATION

This application claims the benefit of and the priority to U.S. Provisional Patent Application No. 63/275,189 filed on Nov. 3, 2021, the entirety of each of which is incorporated by reference in its entirety.

### FIELD OF INVENTION

The present disclosure generally relates to systems and methods for blood pressure monitoring. More specifically, the present disclosure relates to systems and methods for cuff-less blood pressure estimation from photoplethysmography via machine learning modeling.

### BACKGROUND OF THE INVENTION

Hypertension, or high blood pressure (BP), is a wide-spread public health challenge that can cause cardiovascular diseases such as cardiomyopathy, as well as damages to brain and kidney leading to stroke and diabetes.

### SUMMARY OF THE INVENTION

The summary is a high-level overview of various aspects of the invention and introduces some of the concepts that are further detailed in the Detailed Description section below. This summary is not intended to identify key or essential features of the claimed subject matter, nor is it intended to be used in isolation to determine the scope of the claimed subject matter. The subject matter should be understood by reference to the appropriate portions of the entire specification, any or all drawings, and each claim.

Embodiments of the present disclosure relate to a computer-implemented method including receiving, by at least one processor, a plurality of digital signal data from at least one sensor device of a plurality of sensor devices. The method also includes dynamically converting, by the at least one processor, the plurality of digital signal data into at least one visibility point in a plurality of visibility points based on at least one time series vector of a plurality of time series vectors associated with the plurality of digital signal data. The method also includes generating, by the at least one processor, utilizing the at least one visibility point, at least one image to preserve the at least one time series vector associated with the plurality of digital signal data, where the at least one time series vector is a shape within the at least one image. The method also includes extracting, by the at least one processor, at least one feature metric of a plurality of feature metrics from the at least one image based on an analysis of a pre-trained machine learning algorithm. The method also includes automatically determining, by the at least one processor, utilizing a transfer learning algorithm, a first position of at least one node in a plurality of nodes within the at least one image based on a relationship between the at least one feature metric and the at least one time series vector associated with the plurality of digital time series data. The method also includes predicting, by the at least one processor, utilizing the transfer learning algorithm, a second position of the at least one node in the plurality of nodes based on the analysis of the pre-trained machine learning algorithm and the relation between the at least one feature metric and the at least one time series vector.

In some embodiments, the plurality of digital signal data includes a plurality of physiological data signals.

In some embodiments, the at least one visibility point preserves a plurality of temporal information associated with a data waveform based on the plurality of digital signal data.

In some embodiments, the pre-trained machine learning algorithm includes a deep conventional neural network.

In some embodiments, predicting the second of the at least one node in the plurality of nodes includes estimating a systolic blood pressure and a diastolic blood pressure associated with at least one user in a plurality of users based on the plurality of feature metrics and the plural of time series vectors.

In some embodiments, the pre-trained learning algorithm is one of AlexNet, VGG-19 or Inception v3.

In some embodiments, the plurality of digital signal data includes PPG data and reference BP data.

In some embodiments, the at least one image is a visibility graph (VG).

In some embodiments, the method further includes applying a selection procedure to the plurality of digital signal data to remove at least one of: duplicated digital signal data, digital signal data of poor quality, or digital signal with less than a predetermined number of systolic peaks.

In some embodiments, the method further includes classifying the feature metric into one of at least two classes.

Embodiments of the present disclosure also relate to a system including at least one processor configured to execute software instructions, where the software instructions, when executed, cause the at least one processor to perform steps to: receive a plurality of digital signal data from at least one sensor device of a plurality of sensor devices. The processor is also caused to dynamically convert the plurality of digital signal data into at least one visibility point in a plurality of visibility points based on at least one time series vector of a plurality of time series vectors associated with the plurality of digital signal data. The processor is also caused to generate, utilizing the at least one visibility point, at least one image to preserve the at least one time series vector associated with the plurality of digital signal data, where the at least one time series vector is a shape within the at least one image. The processor is also caused to extract at least one feature metric of a plurality of feature metrics from the at least one image based on an analysis of a pre-trained machine learning algorithm. The processor is also caused to automatically determine, utilizing a transfer learning algorithm, a first position of at least one node in a plurality of nodes within the at least one image based on a relationship between the at least one feature metric and the at least one time series vector associated with the plurality of digital time series data. The processor is also caused to predict, utilizing the transfer learning algorithm, a second position of the at least one node in the plurality of nodes based on the analysis of the pre-trained machine learning algorithm and the relation between the at least one feature metric and the at least one time series vector.

In some embodiments, the plurality of digital signal data includes a plurality of physiological data signals.

In some embodiments, the at least one visibility point preserves a plurality of temporal information associated with a data waveform based on the plurality of digital signal data.

In some embodiments, the pre-trained machine learning algorithm includes a deep conventional neural network.

In some embodiments, the processor is also caused to predict the second of the at least one node in the plurality of nodes includes estimating a systolic blood pressure and a diastolic blood pressure associated with at least one user in a plurality of users based on the plurality of feature metrics and the plural of time series vectors.

In some embodiments, the pre-trained learning algorithm is one of AlexNet, VGG-19 or Inception v3.

In some embodiments, the plurality of digital signal data includes PPG data and reference BP data.

In some embodiments, the at least one image is a visibility graph (VG).

In some embodiments, the software instructions, when executed, further cause the at least one processor to perform steps to apply a selection procedure to the plurality of digital signal data to remove at least one of: duplicated digital signal data, digital signal data of poor quality, or digital signal data with less than a predetermined number of systolic peaks.

In some embodiments, the software instructions, when executed, further cause the at least one processor to perform steps to classify the feature metric into one of at least two classes.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the present disclosure. The patent or application file contains at least one drawing executed in color. Copies of this patent or patent application publication with color drawing(s) will be provided by the Office upon request and payment of the necessary fee.

FIG. 1 is a block diagram illustrating an operating computer architecture for cuff-less blood pressure estimation from photoplethysmorgraphy (PPG) via visibility graph (VG) and transfer learning, according to one or more embodiments of the present disclosure.

FIG. 2 is a flow diagram depicting the proposed method for estimating blood pressure (BP) from PPG via VG and transfer learning, according to embodiments of the present disclosure.

FIG. 3 is a graph depicting systolic peaks extracted from a PPG signal, according to embodiments of the present disclosure.

FIG. 4 is a graph depicting extracted 1-beat windows, according to embodiments of the present disclosure.

FIG. 5 is a graph depicting extracted 3-peak windows, according to embodiments of the present disclosure.

FIG. 6 is a graph depicting VG mapping of the shape of a waveform into the connectives of nodes in VG, according to embodiments of the present disclosure.

FIG. 7 is a graph depicting a PPG signal containing one cardiac cycle, according to embodiments of the present disclosure.

FIG. 8 is a graph depicting a color-coded VG adjacency matrix corresponding to the graph of FIG. 7, according to embodiments of the present disclosure.

FIG. 9 is a graph depicting a PPG signal under zero-padded 3-peak setting, according to embodiments of the present disclosure.

FIG. 10 is an inverted graph of FIG. 9, according to embodiments of the present disclosure.

FIG. 11 is a VG image of a PPG signal corresponding to FIG. 9, according to embodiments of the present disclosure.

FIG. 12 is a VG image of the inverted PPG signal of FIG. 10, according to embodiments of the present disclosure.

FIG. 13 is a flow diagram depicting a transfer learning process, leveraging Inception v3, VGG-19 and AlexNet networks, according to embodiments of the present disclosure.

FIG. 14 is a graph depicting accumulated explained variance over a number of features from PCA, according to embodiments of the present disclosure.

FIG. 15 is a 2-dimensional visualization of flattened VG_{POS} feature vectors, created under 3-peak setting for “normal BP” and “high BP” classes, using t-SNE, according to embodiments of the present disclosure.

FIG. 16 is a regression plot between reference and estimated BP values, according to embodiments of the present disclosure.

FIG. 17 is a Bland-Altman plot of estimated systolic blood pressure (SBP), according to embodiments of the present disclosure.

FIG. 18 is a Bland-Altman plot of estimated diastolic blood pressure (DBP), according to embodiments of the present disclosure.

FIG. 19 is an exemplary structure of an LSTM model, according to embodiments of the present disclosure.

### DETAILED DESCRIPTION

The following description provides exemplary embodiments only, and is not intended to limit the scope, applicability, or configuration of the disclosure. Rather, the following description of the exemplary embodiments will provide those skilled in the art with an enabling description for implementing one or more exemplary embodiments. It will be understood that various changes may be made in the function and arrangement of elements without departing from the spirit and scope of the presently disclosed embodiments. Embodiment examples are described as follows with reference to the figures. Identical, similar, or identically acting elements in the various figures are identified with identical reference numbers and a repeated description of these elements is omitted in part to avoid redundancies.

Among those benefits and improvements that have been disclosed, other objects and advantages of this invention will become apparent from the following description taken in conjunction with the accompanying figures. Detailed embodiments of the present invention are disclosed herein; however, it is to be understood that the disclosed embodiments are merely illustrative of the invention that may be embodied in various forms. In addition, each of the examples given in connection with the various embodiments of the invention which are intended to be illustrative, and not restrictive.

Throughout the specification and claims, the following terms take the meanings explicitly associated herein, unless the context clearly dictates otherwise. The phrases “in one embodiment” and “in some embodiments” as used herein do not necessarily refer to the same embodiment(s), though it may. Furthermore, the phrases “in another embodiment” and “in some other embodiments” as used herein do not necessarily refer to a different embodiment, although it may. Thus, as described below, various embodiments of the invention may be readily combined, without departing from the scope or spirit of the invention.

In addition, as used herein, the term “or” is an inclusive “or” operator and is equivalent to the term “and/or,” unless the context clearly dictates otherwise. The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.”

The term “based on” is not exclusive and allows for being based on additional factors not described, unless the context clearly dictates otherwise. In addition, throughout the specification, the meaning of “a,” “an,” and “the” include plural references. The meaning of “in” includes “in” and “on.” Spatial or directional terms, such as “left”, “right”, “inner”, “outer”, “above”, “below”, and the like, are not to be considered as limiting as the invention can assume various alternative orientations. All numbers used in the specification are to be understood as being modified in all instances by the term “about”. The term “about” means a range of plus or minus ten percent of the stated value.

Hypertension, or high blood pressure (BP), is a wide-spread public health challenge that can cause cardiovascular diseases such as cardiomyopathy, as well as damages to brain and kidney leading to stroke and diabetes. It has been shown that frequent monitoring of BP, self-taken by the patient, enhances patient's long-term adherence and compliance to clinical advice. However, studies have shown that only less than 30% of patients with a BP monitoring device at home have measured their blood pressure more than twice a day, and the frequency of BP measurements varies across individuals. A problem originates from the intrinsic inconvenience of conventional cuff-based blood pressure monitors such as sphygmomanometer and oscillometry-based devices, which cannot continuously record BP due to discomfort of the cuff, the requirement of manual operation for measurement initialization, and the prolonged interval analyzing the oscillometry pressure waveform for obtaining the SBP and DBP readings. Therefore, continuous and non-invasive methods for BP monitoring are highly desirable for treating hypertension.

Continuous BP monitoring may also be critical for groups of patients other than those with hypertension. For example, in patients with spinal cord injury (SCI), an abrupt rise in BP (in excess of 20 mmHg SBP) caused by autonomic dysreflexia (AD) (an autonomic reflex response to nociceptive stimuli), could lead to disabling headache, seizure, cerebral hemorrhage, and even death. As such, accurate and continuous monitoring of BP is critical for early detection of episodes of AD in these patients, to enable the patients to seek timely medical treatments. However, conventional BP monitors are not usable for detecting episodes of AD, again due to the slowness of measurement and the need of manual operation.

Over the past two decades, several BP-estimation approaches, either model-driven or data-driven, have been investigated to enable continuous, cuff-less and non-invasive BP monitoring. Model-driven methods are established based on the relationship between BP and pulse wave velocity (PWV). These methods, however, require at least two signals (e.g., electrocardiogram (ECG) and PPG) as well as subject-specific model parameter calibration that does not hold over time. Data-driven methods address some of the limitations of the model-driven methods. For example, the data-driven methods aim to learn the relationship between the PPG and SBP/DBP via machine learning as there exist high correlation between PPG and the arterial BP in temporal and spectral domains. Thus, the data-driven methods eliminate the need for secondary signal that is required in model-driven methods.

In conventional data-driven methods, either features are manually defined, which typically require an exhaustive process of searching for and identifying the appropriate type and number of features, or the feature-learning capability of deep neural networks (DNNs) is used to find the appropriate features in PPG for BP estimation. While in the DNNs approach, the need for manual feature engineering is eliminated, the requirement of having a large number of parameters to be trained in the model and the large size training set enforces high computational cost.

Development of deep convolutional neural network (CNN) such as AlexNet, VGG and Inception has brought breakthroughs to the field of image recognition. Moreover, the transferability of DNNs makes “transfer learning” possible, in which the weights in the convolutional layers of a pre-trained network can be re-used, leaving only the last few layers in the network needing to be trained on a new dataset, thereby, making it possible to use DNNs on small-scale datasets. Transfer learning has shown promising results in addressing clinical problems involving interpretation of images such as diagnosis of lung diseases, liver cancer, and colorectal polyps.

To enable transfer learning with pre-trained image classification CNNs for problems in which the inputs are time series (rather than images), the time series need to be converted into images. This is a challenging problem, as the conversion should be made in such a way that the created images preserve the information of the time series that aid in classification. Few promising studies, using conversion tools such as the recurrent plot (RP), the gramian angular summation/difference fields (GASF/GADF) and the Markov transition fields (MTF), have recently explored this problem. These include classifying sleep stages from electroencephalography (EEG) signals and diagnosing epilepsy with EEG. However, to the best of our knowledge, no study has offered a solution for the use of transfer learning via image classification networks for the BP-estimation problem.

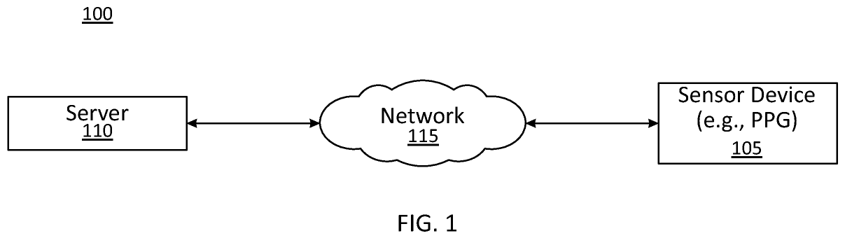

FIGS. 1 to 19 illustrate systems and methods for fast, data-driven deep-learning-based end-to-end estimation of BP from short duration of PPG signals with low computational budgets. The following embodiments provide technical solutions and technical improvements that overcome technical problems, drawbacks and/or deficiencies in at least one technical field involving efficient, accurate and continuous monitoring of BP without the use of BP cuffs. As explained in more detail below, the present disclosure provides a technically advantageous computer architecture that reduces computational budget using transfer learning by leveraging image classification CNNs pre-trained on the ImageNet database. In some embodiments, the systems and methods are technologically improved by enabling the use of pre-trained image classification CNNs for BP estimation by using visibility graph (VG) to convert PPG signals into images. In some embodiments, while conventional DNN-based BP-estimation methods require the training of all the model parameters from scratch, systems and methods of the present disclosure only require the training of one dense layer, and hence, the computational power is reduced. In some embodiments, BP is estimated using only a very short PPG duration (less than 2 s) as an input. In some embodiments, systems and method of the present disclosure are thus capable of capturing rapid, intermittent BP changes, which is a major advantage over conventional method which require longer PPG windows in applications such as detection of early episodes of AD. In some embodiments, the adjacency matrix of VG may be used as an image. In some embodiments, VG may be used to create images that preserve the key temporal information of the PPG wave form, required for BP estimation. Moreover, various practical applications of the disclosed technology are also described, which provide further practical benefits to users and operators that are also new and useful improvements in the art.

FIG. 1 is a block diagram illustration of an exemplary BP estimation system 100 used to implement one of more embodiments of the present disclosure. The components and arrangements of FIG. 1 are not intended to limit the disclosed embodiments as the components are used to implement the disclosed processes and featured may vary. In accordance with disclosed embodiments, the BP estimation system 100 may include a server 110 in communication with at least one sensor device 105 via a network 115.

Network 105 may be of any suitable type, including individual connections via the internet such as cellular or Wi-Fi networks. In some embodiments, network 105 may connect participating devices using direct connections such as radio-frequency identification (RFID), near-field communication (NFC), Bluetooth™, low-energy Bluetooth™ (BLE), Wi-Fi™, ZigBee™ ambient backscatter communications (ABC) protocols, USB, or LAN. Because the information transmitted may be personal or confidential, security concerns may dictate one or more of these types of connections be encrypted or otherwise secured. In some embodiments, however, the information being transmitted may be less personal, and therefore the network connections may be selected for convenience over security.

Server 110 may be associated with a medical institution. For example, server 110 may manage individual patient or practitioner accounts. One of ordinary skill will recognize that server 110 may include one or more logically or physically distinct systems.

In some embodiments, the server 110 may include hardware components such as a processor (not shown), which may execute instructions that may reside in local memory and/or transmitted remotely. In some embodiments, the processor may include any type of data processing capacity, such as a hardware logic circuit, for example, an application specific integrated circuit (ASIC) and a programmable logic, or such as a computing device, for example a microcomputer or microcontroller that includes a programmable microprocessor.

Examples of hardware components may include one or more processors, microprocessors, circuits, circuit elements (e.g., transistors, resistors, capacitors, inductors, and so forth), integrated circuits, application specific integrated circuits (ASIC), programmable logic devices (PLD), digital signal processors (DSP), field programmable gate array (FPGA), logic gates, registers, semiconductor device, chips, microchips, chip sets, and so forth. In some embodiments, the one or more processors may be implemented as a Complex Instruction Set Computer (CISC) or Reduced Instruction Set Computer (RISC) processors; x86 instruction set compatible processors, multi-core, or any other microprocessor or central processing unit (CPU). In various implementations, the one or more processors may be dual-core processor(s), dual-core mobile processor(s), and so forth.

In some embodiments, the at least one sensor device 105 may be any type of sensor device configured to measure blood pressure. In some embodiments, the at least one sensor device 105 may be a PPG, an ECG or an ABP device. In some embodiments, the at least one sensor device 105 is configured to output digital signal data. In some embodiments, the at least one sensor device 105 may output a digital signal record having a length of from 2 seconds to 600 seconds. In some embodiments, the output digital signal record has a length of from 10 seconds to 600 seconds. In some embodiments, the output digital signal record has a length of from 50 seconds to 600 seconds. In some embodiments, the output digital signal record has a length of from 100 seconds to 600 seconds. In some embodiments, the output digital signal record has a length of from 200 seconds to 600 seconds. In some embodiments, the output digital signal record has a length of from 300 seconds to 600 seconds. In some embodiments, the output digital signal record has a length of from 400 seconds to 600 seconds. In some embodiments, the output digital signal record has a length of from 500 seconds to 600 seconds.

In some embodiments, the output digital signal record has a length of from 2 seconds to 500 seconds. In some embodiments, the output digital signal record has a length of from 2 seconds to 400 seconds. In some embodiments, the output digital signal record has a length of from 2 seconds to 300 seconds. In some embodiments, the output digital signal record has a length of from 2 seconds to 200 seconds. In some embodiments, the output digital signal record has a length of from 2 seconds to 100 seconds. In some embodiments, the output digital signal record has a length of from 2 seconds to 50 seconds. In some embodiments, the output digital signal record has a length of from 2 seconds to 10 seconds.

In some embodiments, the output digital signal record has a length of from 8 seconds to 592 seconds. In some embodiments, the output digital signal record has a length of from 70 seconds to 400 seconds. In some embodiments, the output digital signal record has a length of from 200 seconds to 300 seconds. In some embodiments, the output digital signal record has a length of from 425 seconds to 525 seconds. In some embodiments, the output digital signal record has a length of from 150 seconds to 475 seconds. In some embodiments, the output digital signal record has a length of from 350 seconds to 400 seconds. In some embodiments, the output digital signal record has a length of from 100 seconds to 550 seconds.

In some embodiments, the PPG and reference BP signals may be the input into the algorithm. In some embodiments, the reference BP signal may be used to define the reference SBP and DBP values in the training and testing phases. In some embodiments, PPG and reference BP signals may be segmented into non-overlapping windows with respect to the position of the cardiac cycles. In some embodiments, PPG signals within the identified windows may be converted into VGs. In some embodiments, the VGs may then be used as the input to pre-trained CNNs, and a feature vector may be obtained for each VG via forward propagation. In some embodiments, in the training phase, ridge regression may be used to obtain the weights and bias between the feature vectors and the reference BP values. In some embodiments, in the testing phase, new PPG signals may be used as the input and the obtained weights and bias from the training phase may be applied to the feature vector from the output of CNN to find the estimates for SBP and DBP. In some embodiments, the estimated BPs may then be compared to the reference BP values to evaluate the performance of the proposed model.

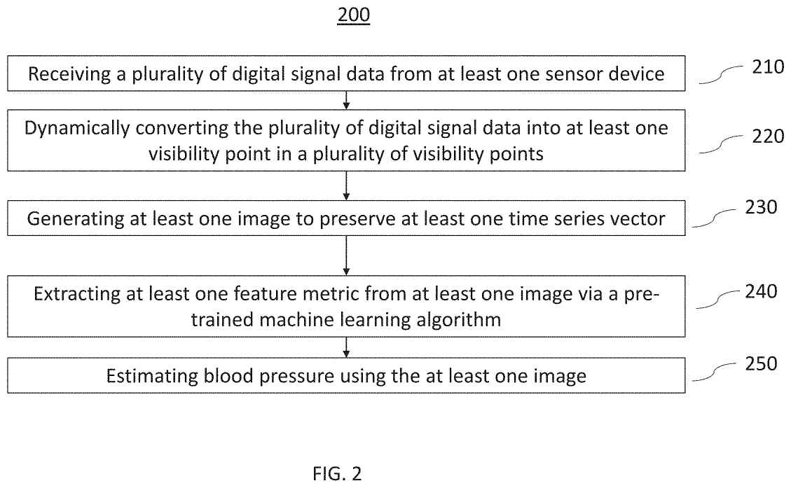

FIG. 2 is a process flow diagram illustration of an example of an illustrated computer-mediated process for cuff-less blood pressure estimation from PPG via VG and transfer learning, according to one or more embodiments of the present disclosure according to one or more embodiments of the present disclosure. The exemplary computer-mediated process 200 may be executed by software, hardware, or a combination thereof. For example, process 200 may be performed by including one or more components described in the BP estimation system 100 of FIG. 1 (e.g., server 110, at least one sensor device 105 and network 115).

At 210, digital signal data may be received and processed using a selection procedure to determine acceptable records. In some embodiments, the digital signal data may include digital signal records obtained by the at least one sensor device 105. In some embodiments, the selection procedure may remove duplicated digital signal data records. In some embodiments, each digital signal data record may be divided into non-overlapping segments of 10-second duration. In some embodiments, the 10-second duration may be selected to provide sufficient length for a windowing process, which will be described in further detail below. In some embodiments, the duration may be 5 seconds to 15 seconds. In some embodiments, the duration may be 7 seconds to 15 seconds. In some embodiments, the duration may be 9 seconds to 15 seconds. In some embodiments, the duration may be 11 seconds to 15 seconds. In some embodiments, the duration may be 13 seconds to 15 seconds.

In some embodiments, the duration may be 5 seconds to 13 seconds. In some embodiments, the duration may be 5 seconds to 11 seconds. In some embodiments, the duration may be 5 seconds to 9 seconds. In some embodiments, the duration may be 5 seconds to 7 seconds.

In some embodiments, the duration may be 7 seconds to 13 seconds. In some embodiments, the duration may be 9 seconds to 13 seconds. In some embodiments, the duration may be 11 seconds to 15 seconds. In some embodiments, the duration may be 11 seconds to 13 seconds. In some embodiments, the duration may be 7 seconds to 11 seconds. In some embodiments, the duration may be 9 seconds to 11 seconds. In some embodiments, the duration may be 7 seconds to 9 seconds.

In some embodiments, segments containing saturated amplitudes may be removed. In some embodiments, segments with discontinuity in the PPG signal may be removed.

In some embodiments, segments with less than 10 detectable systolic peaks may be removed. In some embodiments, segments with less than 9 detectable systolic peaks may be removed. In some embodiments, segments with less than 8 detectable systolic peaks may be removed. In some embodiments, segments with less than 7 detectable systolic peaks may be removed. In some embodiments, segments with less than 6 detectable systolic peaks may be removed. In some embodiments, segments with less than 5 detectable systolic peaks may be removed. In some embodiments, systolic peak detection may be performed using the algorithm proposed in M. Elgendi, I. Norton, M. Brearley, D. Abbott, and D. Schuurmans, “Systolic peak detection in acceleration photoplethysmograms measured from emergency responders in tropical conditions,” PLOS One, vol. 8, no. 10, p. e76585, 2013.

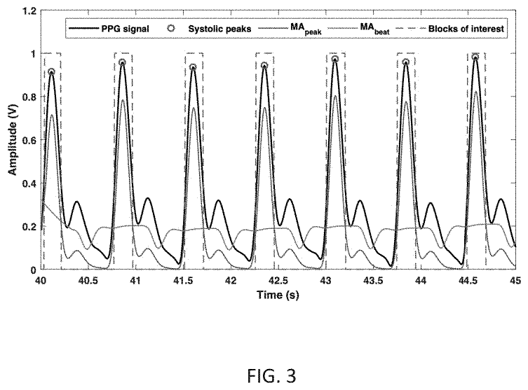

In some embodiments, using this algorithm, first, the square of the amplitude of each sample in the PPG signal may be calculated. In some embodiments, two moving average filters, namely MApeak and MAbeat, are then applied, creating two curves. An exemplary graph depicting extracted systolic peaks from a PPG signal is shown in FIG. 3. In some embodiments, the intervals with larger amplitude for MApeak curve compared to MAbeat are identified (shown in FIG. 3 as blocks of curves interest), as they indicate the presence of systolic peaks. Considering normal human heart rate to be above 60 beats per minute, segments of 10-second duration should have more than 10 detectable systolic peaks. As the algorithm may not be able to detect the first and/or last systolic peak in the segment due to lack of existence of complete cardiac cycles at the start/end of the segment, in some embodiments, the threshold of removal may be selected to be 8 systolic peaks.

In some embodiments, segments with inconsistencies, in which significant changes exist in the baseline, amplitude or cycle duration, that could be the results of signal corruptions are removed. In some embodiments, to identify these segments, in each segment, the mean and standard deviation of the amplitude of the systolic peaks, the amplitude of turning points, and the PPG cycle duration may be computed. In some embodiments, segments with outliers outside of the boundary of mean 3 standard of the computed values are then removed.

Finally, in some embodiments, records with poor overall quality may be removed using a strategy suggested by X. Xing, Z. Ma, M. Zhang, Y. Zhou, W. Dong, and M. Song, “An unobtrusive and calibration-free blood pressure estimation method using photoplethysmography and biometrics,” Scientific Reports, vol. 9, no. 1, p. 8611, 2019. For example, in some embodiments, if from the above steps more than 30% of the segments in a record are removed, then all segments belonging to that record were removed.

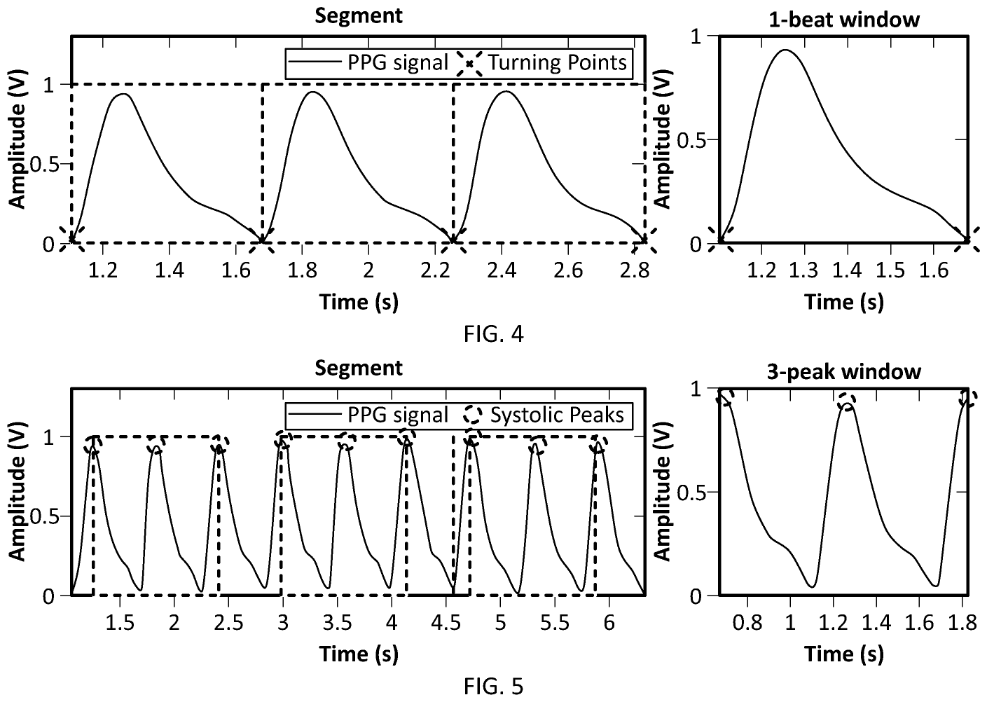

At step 220, the digital signal data may be converted into at least one visibility point in a plurality of visibility points. Specifically, the digital signal segments may be divided into non-overlapping beat-to-beat windows for estimating BP. In some embodiments, two settings for windowing may implemented: 1-beat and 3-peak settings. In some embodiments, for the 1-beat setting, each window contains one complete cardiac cycle of the PPG signal. In some embodiments, for the 3-peak setting, each window includes 3 consecutive systolic peaks, consisting of one complete cardiac cycle plus part of the previous and next cycles. In some embodiments, in both settings, the created windows may not share signals from the same cardiac cycle, to ensure that there is no leakage between the training and testing sets. FIG. 4 is a graph illustrating a 1-beat window method for part of a segment, according to embodiments of the present disclosure. FIG. 5 is a graph illustrating a 3-peak window method for part of a segment, according to embodiments of the present disclosure. In some embodiments, the amplitude of the PPG signal in each window may be remapped between 0 and 1. In some embodiments, to find the reference BP values from the ABP signal for each PPG window, the corresponding ABP window may be located with the same starting and ending time instances. In some embodiments, the maximum and minimum amplitude of the ABP signal within the window may then be used as the reference SBP and DBP values, respectively, for the corresponding PPG window.

In some embodiments the windows may be used to train the transfer learning model. In some embodiments, 85% of the windows my be used to train the transfer learning model. In some embodiments, 75% to 95% of the windows my be used to train the transfer learning model. In some embodiments, 80% to 95% of the windows my be used to train the transfer learning model. In some embodiments, 85% to 95% of the windows my be used to train the transfer learning model. In some embodiments, 90% to 95% of the windows my be used to train the transfer learning model.

In some embodiments, 75% to 90% of the windows my be used to train the transfer learning model. In some embodiments, 75% to 85% of the windows my be used to train the transfer learning model. In some embodiments, 75% to 80% of the windows my be used to train the transfer learning model.

In some embodiments, 80% to 90% of the windows my be used to train the transfer learning model. In some embodiments, 80% to 85% of the windows my be used to train the transfer learning model. In some embodiments, 85% to 90% of the windows my be used to train the transfer learning model.

In some embodiments, the windows may be used to test the transfer learning model. In some embodiments, 15% of the windows may be used to test the transfer learning model. In some embodiments 10% to 30% of the windows may be used to test the transfer learning model. In some embodiments 15% to 30% of the windows may be used to test the transfer learning model. In some embodiments 20% to 30% of the windows may be used to test the transfer learning model. In some embodiments 25% to 30% of the windows may be used to test the transfer learning model.

In some embodiments 10% to 25% of the windows may be used to test the transfer learning model. In some embodiments 10% to 20% of the windows may be used to test the transfer learning model. In some embodiments 10% to 15% of the windows may be used to test the transfer learning model.

In some embodiments 15% to 25% of the windows may be used to test the transfer learning model. In some embodiments 15% to 20% of the windows may be used to test the transfer learning model. In some embodiments 20% to 25% of the windows may be used to test the transfer learning model.

At step 230, at least one image is generated to preserve the at least one time series vector. Specifically, in some embodiments, images from PPG waveforms may be generated using VG. In some embodiments, VG may transform a given time series into an undirected graph by inspecting the natural visibility of the samples' amplitudes. In some embodiments, VG may be used for classification problems involving physiological signals, where various graph measures extracted from VG have been used as features.

In some embodiments, x=[x1, . . . , x_{L}] may represent a time series of L points, where xᵢ (i=1, . . . , L) denotes the i^{th} sample in the time series. In some embodiments, tᵢ may represent the time corresponding to the occurrence of sample xᵢ. In some embodiments, in order to construct the visibility graph for this time series, each sample may be considered as a node in the graph. In some embodiments, an undirected edge may be formed between any two nodes if they are considered to be naturally visible. For example, in some embodiments, for two nodes h and l (tₕ<tᵢ), there will be an undirected and unweighted edge if.

$${x}_{p} < {x}_{l} + ( {x}_{h} - {x}_{l} ) \frac{{t}_{l} - {t}_{p}}{{t}_{l} - {t}_{h}} , ∀ p ∈ \{ p \vert {t}_{h} < {t}_{p} < {t}_{l} \} . \qquad ( Eq . 1 )$$

FIG. 6 depicts how VG may map the shape of a waveform into the connectives of nodes in VG, according to embodiments of the present disclosure. In some embodiments, the visibility of any two nodes may be visualized by drawing a line between their corresponding amplitudes. In some embodiments, if the drawn line does not intersect the amplitudes of the nodes between them, then the two nodes are considered as naturally visible. For example, in FIG. 6, for Node 2, examples of visible and not visible nodes (such as Node 52) are shown with blue and black circles, respectively. In some embodiments, in its corresponding visibility graph, these visible nodes may be connected by edges. As such, in some embodiments, the shape of the waveform may be encoded into the connectives between the nodes in VG.

Conventional VG-based classification studies only extract specific graph measures from the constructed VG and use them as features. However, in some embodiments, systems and methods of the present disclosure take a data-driven approach, and may use VG's adjacency matrix, which contains the temporal information, as a 1-channel gray-scale image for input to pre-trained image classification CNNs.

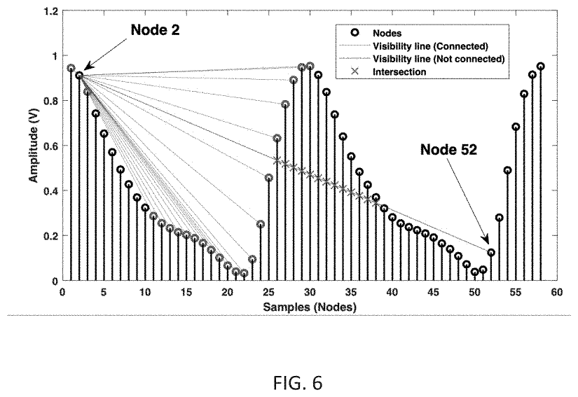

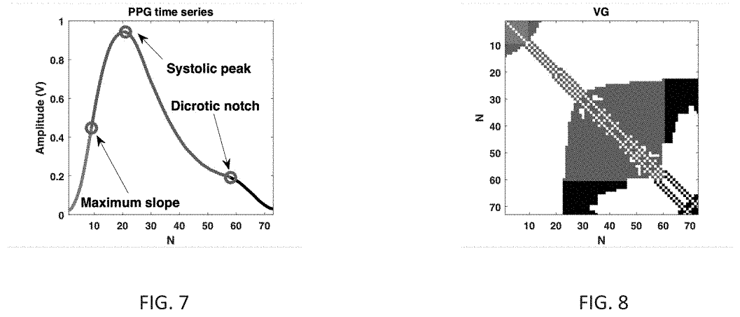

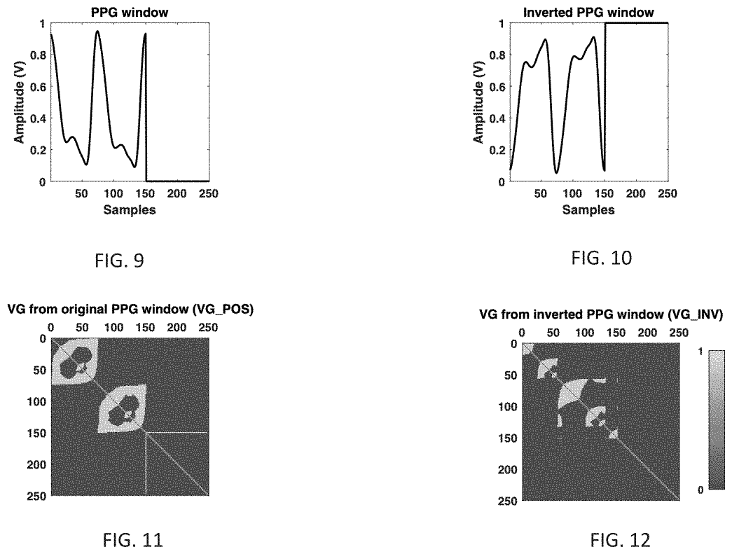

FIG. 7 is a graph displaying the PPG signal from one cardiac cycle (1-beat window setting), according to embodiments of the present disclosure. FIG. 8 is a color-coded VG adjacency matrix corresponding to FIG. 7, according to embodiments of the present disclosure. As can be seen in FIG. 8, the position of the PPG maximum slope may be expressed as the size of the top-left triangle-like shape, and the duration between the systolic peak and the dicrotic notch is expressed as the size of the red sector, suggesting that the created images contain the temporal-domain features of the PPG signal. In some embodiments, a different VG image may be created from the same window by inverting the waveform, as shown in FIGS. 9-12. For example, FIG. 9 is a graph depicting a PPG signal under zero-padded 3-peak setting while FIG. 10 is a graph depicting the inverted version of the PPG signal of FIG. 9. Additionally, FIG. 11 is a VG image corresponding to the PPG signal of FIG. 9 while FIG. 12 is a VG image corresponding to the inverted PPG signal of FIG. 10. In some embodiments, the VG created from the PPG waveform may be referred to as VG_{POS} (e.g., FIG. 11), and the VG created from the inverted PPG waveform may be referred to as VG_{INV} (e.g., FIG. 12).

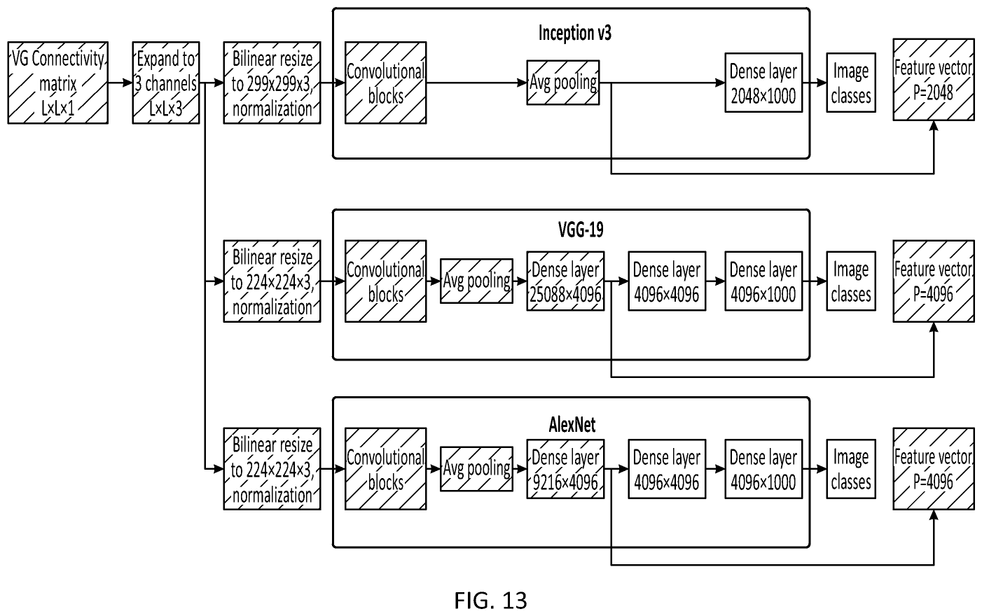

At step 240, at least one feature metric is extracted using a pre-trained machine learning algorithm. Specifically, in some embodiments, using transfer learning, the VGs are input into pre-trained CNNs and a feature vector is obtained for each VG via forward propagation. In some embodiments, for transfer learning, three well-known pre-trained CNNs (Inception v3, VGG-19, and AlexNet, with 48, 19 and 8 layers, respectively) may be implemented. FIG. 13 illustrates the details of each of these three models. As depicted in FIG. 13, yellow blocks indicate activated layers in the forward propagation process. In some embodiments, because all of the considered CNN models take 3-channel Red-Green-Blue (RGB) images as their input, each VG image may first be extended to 3 channels by replicating itself, being interpreted as a gray-scale image by CNN. Next, in some embodiments, the VG images may be bilinearily resized to the desired input shapes of each CNN, and passed as inputs to the pre-trained models.

In some embodiments, by forward propagation, a P-dimensional feature vector v∈R^{P} may be obtained for each VG image. In some embodiments, for the three models, v may be the input of the final dense layer for Inception v3, and the output of the first dense layer for VGG-19 and AlexNet. In some embodiments, since both VG_{POS} and VG_{INV} were created for each PPG window, two feature vectors, v_{POS} and v_{INV}, may be obtained. As such, in some embodiments, the BP-estimation problem may be interpreted as a regression problem between the feature vector and the reference SBP and DBP values.

In some embodiments, to estimate each pair of SBP and DBP, linear weights (w) and bias (b) may be solved for using the ridge regression method. For example, in some embodiments, an assumption may be made that N denotes the size of the training set, T denotes transpose, y contains the reference SBP and DBP values

$$( i . e . , y = {[ \begin{matrix}{SBP}_{1} & {SBP}_{2} & … & {SBP}_{N} \\ {DBP}_{1} & {DBP}_{2} & … & {DBP}_{N}\end{matrix} ]}^{T} ) ,$$

\tilde{X} is the matrix containing the feature vectors vᵢ, i=1, . . . , N, (i.e.,

$${[ \begin{matrix}{v}_{1} & {v}_{2} & … & {v}_{n} \\ 1 & 1 & … & 1\end{matrix} ]}^{T} ) ,$$

and λ is the regularization parameter, ridge regression may solve the regularized least-square regression problem:

$${arg min}_{\tilde{w}} {‖ \tilde{X} \tilde{w} - y ‖}^{2} + λ {‖ w ‖}^{2} , \qquad ( Eq . 2 )$$

with a closed-form solution:

\tilde{w}=(λI+\tilde{X}^{T}\tilde{X})⁻¹\tilde{X}^{T}y (Eq. 3)

where I is the identity matrix, an

$$\tilde{w} = [ \begin{matrix}w \\ b\end{matrix} ]$$

to be found. In some embodiments, three different settings may be considered for the feature vector:

$${v}_{i} = {v}_{POSi} , {v}_{i} = {v}_{INVi} , and {v}_{i} = [ \begin{matrix}{v}_{{POS}_{i}} \\ {v}_{{INV}_{i}}\end{matrix} ] ,$$

for i=1, . . . , N.

In some embodiments, the pre-processing, peak detection, window extraction and VG image creation processes may be completed using MATLAB R2019b. In some embodiments, the extraction of feature vectors from VG images with pre-trained AlexNet, VGG-19 and Inception v3 may be completed with PyTorch 1.7.1. In some embodiments, ridge regression may be performed with scikit-learn.

At step 250, VG images may be used as features for BP estimation. In some embodiments, in order to show the feasibility of estimating BP from VG images, the t-distributed stochastic neighbor embedding (t-SNE) method may be used to visualize the clusters formed by VG images corresponding to various BP levels. In some embodiments, t-SNE may be a non-linear unsupervised dimension reduction method that preserves the clustering relationships of the original high-dimensional feature vectors. In some embodiments, principal component analysis (PCA) may be applied to reject noise and accelerate distance calculation between the feature vectors, and then, applied t-SNE to reduce the dimension to 2, such that visualization can be done in a 2-dimensional plane. In some embodiments, in t-SNE, first the pair-wise similarity of high-dimensional features is computed as conditional probabilities, each feature is then mapped into a 2-dimensional plane, and the similarity between projected data points is computed using the student t-distribution. In some embodiments, t-SNE then aims to match the distributions of the similarities obtained in high and low dimensional space using gradient descent method, thereby, similar high-dimensional feature vectors will be represented as a cluster of points in a 2-dimensional plane.

In some embodiments, PPG windows may be obtained from a 3-peak window setting. In some embodiments, the features may be separated into two classes. For example in some embodiments, the classes may be “normal BP” or “high BP”. In some embodiments, the features may be classified as “high BP” if SBP is greater than 140 mmHg or DBP is greater than 90 mmHg. In some embodiments, all other features may be classified as “normal BP”.

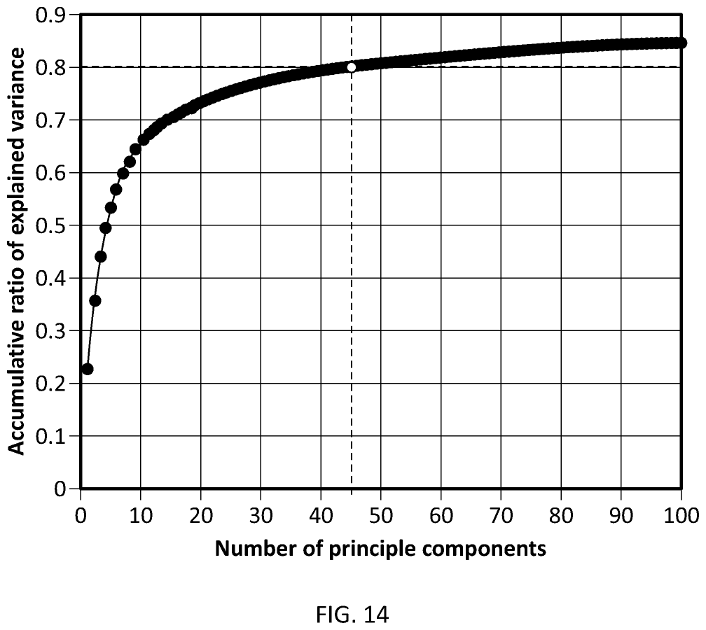

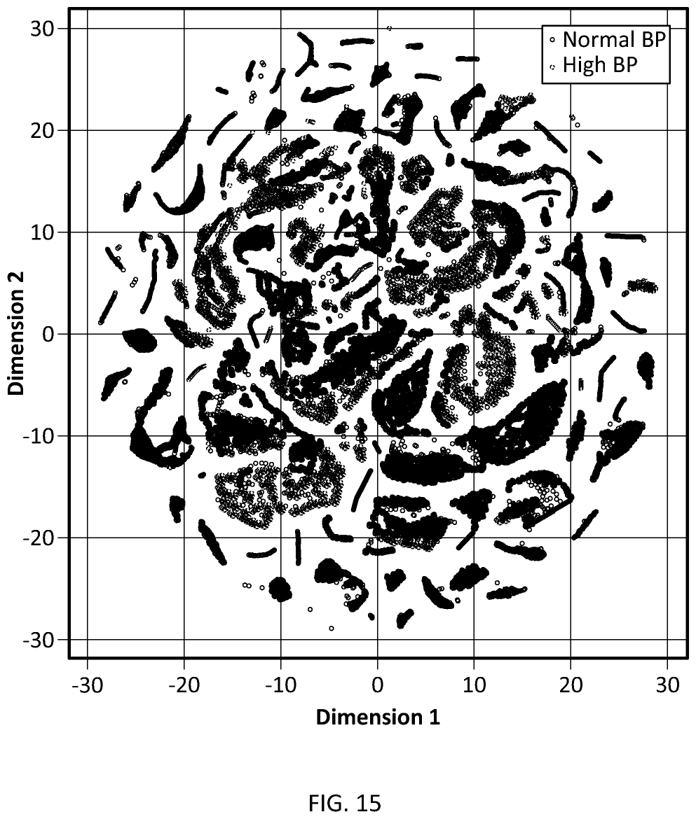

In some embodiments, each VG_{POS} feature may be flattened. In some embodiments, the dimension of the feature vectors may then be reduced by applying principal component analysis (PCA). In some embodiments, a number to be reduced to may be selected based on the number of features that preserves more than 80% of total variance. In some embodiments, t-SNE may be utilized to enable cluster visualization of the feature vectors in a 2-dimensional plane. FIG. 14 is an exemplary graph depicting accumulated explained variance over a number of features from PCA, according to embodiments of the present disclosure. FIG. 15 is an exemplary 2-dimensional visualization of flattened VG_{POS} feature vectors, created under 3-peak window setting, for “normal BP” and “high BP” classes, using t-SNE, according to embodiments of the present disclosure. As can be seen in FIG. 15, in some embodiments, the feature vectors labeled as “high BP” form recognizable clusters, suggesting that VGs generated from PPG windows corresponding to “high BP” have higher similarity to each other than to VGs generated from PPG windows corresponding to “normal BP”.

### Example 1

The method of the present disclosure was tested using a dataset from the University of California Irvine (UCI) Machine Learning Repository, which is a subset of the Multi-parameter Intelligent Monitoring in Intensive Care (MIMIC)-II waveform database. The UCI database contains 12000 records of length ranging from 8 s to 592 s. Each record contained synchronized PPG, ECG, and arterial BP (ABP) measurements, sampled at 125 Hz. The ABP in MIMIC-II database was obtained invasively via cannula needle having direct contact with a radial artery. This method of obtaining the ABP provides instantaneous measures of BP and is considered as the gold standard for BP measurement. Hence, here, an exemplary embodiment may use it as the reference BP.

Using the selection procedure to find acceptable records within the dataset resulted in 11294 segments to work with from 348 records, with the mean and standard deviation of 118.22±18.01 mmHg for SBP values and 64.34±9.66 mmHg for DBP values.

Under 1-beat and 3-peak window segmentation settings, 160721 and 57757 PPG windows were extracted, respectively. Finally, the amplitude of the PPG signal in each window was remapped between 0 and 1. 1-beat windows were one-padded to 125 samples, and 3-peak windows were zero-padded to 250 samples, for unified image conversion.

The 57757 PPG windows obtained from 3-peak window setting were used as features and their corresponding VG_{POS} features of size 250×250 were considered. The features were separated into 2 classes, namely ‘normal BP’ and ‘high BP’: if SBP>140 mmHg or DBP>90 mmHg, then the feature is labeled as ‘high BP’; otherwise it is labeled as ‘normal BP’. This classification resulted in 49250 ‘normal BP’, and 8507 ‘high BP’ features. Each 250×250 VG_{POS} feature were flattened, resulting in a size 62500×1. The dimension of the feature vectors then reduced to 45, by applying PCA. 45 was selected as it was the number of features that preserved more than 80% of total variance. Finally, t-SNE (perplexity=30) was utilized to enable cluster visualization of these 57757 feature vectors in a 2-dimensional plane.

As previously discussed, the exemplary BP estimation system 100 was employed using: two methods for window segmentation (1-beat and the 3-peak), three forms of feature vector (v_{POS}, v_{INV}, and concatenation of v_{POS} and v_{INV}), and three architectures of pre-trained CNNs (Inception v3, VGG-19, and AlexNet). For every combination of these settings, 85% of the available PPG windows were used for training, and 15% were used for testing. In some embodiments, the regularization strength λ used in each combination of settings was selected as a hyper-parameter by grid search using 5-fold cross validation on the training set.

### Example 2: Results

Table 1 summarizes the results for correlation, estimation error and computational time for various settings. It can be seen that, the best results in terms of highest correlation and lowest standard deviation (SD), mean absolute error (MAE) and root mean square error (RMSE) were achieved with pre-trained AlexNet under the 3-peak window segmentation setting and using the concatenated feature vectors of both VG_{POS} and VG_{INV}. In some embodiments, this combination of settings may be considered the optimal setting. Under this setting, an accuracy of −0.00±8.46 for SDP, and −0.04±5.36 mmHg for DBP (mean error (ME) SD) was obtained.

TABLE 1

| Pre-trained | Window | Feature | SBP | | | | DBP | | | | Computational |
|---|---|---|---|---|---|---|---|---|---|---|---|
| Model | Setting | Vector | R | ME ± SD | MAE | RMSE | R | ME ± SD | MAE | RMSE | Time (s) |
| Inception v3 | 1-beat | v_{POS} | 0.75 | −0.04 ± 11.80 | 8.85 | 11.80 | 0.69 | 0.03 ± 7.18 | 5.23 | 7.18 | 0.03 |
| (P = 2048) | (N = 160721) | v_{INV} | 0.76 | −0.04 ± 11.70 | 8.82 | 11.70 | 0.69 | 0.04 ± 7.03 | 5.11 | 7.03 | 0.03 |
| | | Concatenated | 0.80 | −0.03 ± 10.73 | 8.04 | 10.73 | 0.74 | 0.05 ± 6.49 | 4.69 | 6.49 | 0.04 |
| | 3-peak | v_{POS} | 0.78 | −0.05 ± 11.22 | 8.39 | 11.22 | 0.70 | −0.04 ± 7.02 | 5.00 | 7.02 | 0.03 |
| | (N = 57757) | v_{INV} | 0.76 | −0.10 ± 11.74 | 8.77 | 11.74 | 0.70 | −0.02 ± 7.01 | 5.01 | 7.01 | 0.03 |
| | | Concatenated | 0.82 | −0.06 ± 10.34 | 7.66 | 10.34 | 0.76 | −0.03 ± 6.33 | 4.45 | 6.33 | 0.04 |
| VGG-19 | 1-beat | v_{POS} | 0.81 | 0.06 ± 10.511 | 7.78 | 10.51 | 0.76 | 0.03 ± 6.30 | 4.52 | 6.30 | 0.02 |
| (P = 4096) | (N= 160721) | v_{INV} | 0.81 | −0.03 ± 10.52 | 7.76 | 10.52 | 0.76 | 0.03 ± 6.30 | 4.49 | 6.30 | 0.02 |
| | | Concatenated | 0.85 | 0.04 ± 9.58 | 7.08 | 9.58 | 0.80 | 0.03 ± 5.74 | 4.08 | 5.74 | 0.03 |
| | 3-peak | v_{POS} | 0.84 | 0.02 ± 9.73 | 7.15 | 9.73 | 0.77 | 0.10 ± 6.20 | 4.36 | 6.20 | 0.02 |
| | (N = 57757) | v_{INV} | 0.82 | −0.02 ± 10.21 | 7.60 | 10.21 | 0.77 | −0.01 ± 6.21 | 4.36 | 6.21 | 0.02 |
| | | Concatenated | 0.87 | −0.04 ± 8.97 | 6.62 | 8.97 | 0.82 | 0.05 ± 5.64 | 3.93 | 5.64 | 0.03 |
| AlexNet | 1-beat | v_{POS} | 0.84 | −0.02 ± 9.75 | 7.17 | 9.75 | 0.79 | 0.06 ± 5.91 | 4.19 | 5.91 | 0.01 |
| (P = 4096) | (N = 160721) | v_{INV} | 0.83 | 0.00 ± 9.95 | 7.32 | 9.95 | 0.79 | 0.04 ± 5.91 | 4.18 | 5.91 | 0.01 |
| | | Concatenated | 0.87 | −0.03 ± 8.95 | 6.55 | 8.95 | 0.83 | 0.05 ± 5.40 | 3.78 | 5.40 | 0.02 |
| | 3-peak | v_{POS} | 0.86 | −0.04 ± 9.24 | 6.77 | 9.24 | 0.80 | −0.04 ± 5.86 | 4.02 | 5.86 | 0.02 |
| | (N = 57757) | v_{INV} | 0.84 | 0.05 ± 9.68 | 7.04 | 9.68 | 0.80 | 0.00 ± 5.88 | 4.05 | 5.88 | 0.02 |
| | | Concatenated | 0.88 | −0.00 ± 8.46 | 6.17 | 8.46 | 0.84 | −0.04 ± 5.36 | 3.66 | 5.36 | 0.02 |

The results also demonstrate that using the proposed method, DBP estimations generally achieved smaller MAE, RMSE, and SD than SBP estimations under all settings. The DBP performances under all settings are within the limits of the American National Standards of the Association for the Advancement of Medical Instrumentation (AAMI), where the maximum acceptable error is 5±8 mmHg.

Table 1 also indicates that for all 3 types of features (VG_{POS} VG_{INV}, and concatenated) and the 3-peak setting, in most cases provides better SBP and DBP estimation accuracy than the 1-beat setting. As depicted in FIGS. 9-12, the 3-peak setting includes a complete PPG cycle, the diastolic phase of the previous PPG cycle, and the systolic phase of the following PPG cycle. The results suggest that the inclusion of extra information from the previous and following PPG cardiac cycles provides more useful BP-relevant information compared to the 1-beat setting which contains information from only one cardiac cycle.

Under the same window setting, as can be seen in Table 1, comparisons among different types of features show that features extracted from VG_{POS} or VG_{INV} alone gives very close results, while using concatenated feature vectors from both VG_{POS} and VG_{INV} yields better results than using either of them alone. However, performances from using these 3 types of features are close to one another, indicating potential redundancy and co-linearity between the feature vectors extracted from VG_{POS} and VG_{INV}.

As can be seen in Table 1, a comparison among different pre-trained models suggest that while there are some differences in the estimation performance across the models, all deliver reasonably good results (with AlexNet providing the best among the three), suggesting that the proposed PPG-image-creation approach with transfer learning works for BP estimation. AlexNet, the shallowest network among the three, performs the best compared to VGG-19 and Inception v3. This outcome may be related to the larger contribution of the final dense layer in shallow networks (AlexNet) as compared to in deep networks (VGG, Inception v3) in data transformation, as an exemplary embodiment may only fine-tune the last layer.

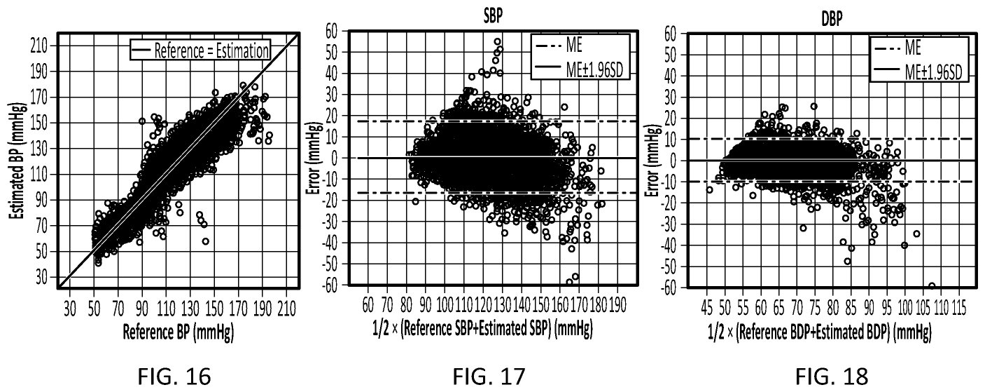

In terms of computational time, once the model is trained, for all cases, it requires less than 50 ms to get the BP estimation from raw PPG input, which indicates that our method is computationally fast. FIG. 16 depicts a regression plot between the reference and estimated BP values obtained from using the optimal setting. FIG. 17 depicts a Bland-Altman plot of estimated SBP and FIG. 18 depicts a Bland-Altman plot for estimated DBP, obtained from using the optimal setting. A comparison of the optimal results to the British Hypertension Society (BHS) protocol is summarized in Table II. Using the optimal setting, the SBP estimation performance ranked grade B under the BHS protocol, while the DBP ranked grade A.

TABLE 2

| | Probability (%) | | |
|---|---|---|---|
| Item | ≤5 mmHg | ≤10 mmHg | ≤15 mmHg |
| SBP | 53.46 | 81.15 | 92.43 |
| DBP | 75.72 | 95.04 | 98.56 |
| Grade A | 60 | 85 | 95 |
| Grade B | 50 | 75 | 90 |
| Grade C | 40 | 65 | 85 |

The results show that, using the systems and methods of the present disclosure, the VG images can be used to build a BP-estimation model that is fast, simple, robust and free of the requirements of manual feature engineering. The systems and methods of the present disclosure are capable of capturing rapid, intermittent BP changes, which is a major advantage over methods requiring longer PPG windows in applications such as detection of early episodes of AD.

### Example 3

Table 3 compares the disclosed method with PPG-based BP estimation studies, in terms of estimation performance, required duration of PPG, feature extraction (data driven vs manual) and model complexity. Very few had reported the Pearson's correlation coefficient (R) metric, so only the ME or MAE results have been included in Table 3. As there are differences in the dataset, method implementation and validation procedures across studies, a fair quantitative comparison cannot be made. In addition, when making the comparison, trade-offs between performance, speed, and computational complexity measures should be taken into account.

TABLE 3

| | | Required Input | Required Parameter | Feature | Performance | |
|---|---|---|---|---|---|---|
| Citation | Dataset | PPG Duration | Training | Extraction | SBP | DBP |
| [symbol] work | MIMIC-II [symbol] | ≤2 [symbol] | Ridge regression | Data Driven, | −0.00 ± 8.40 | −0.04 + 5.36 |
| | | | | VG creation | (ME ± SD) | (ME ± SD) |
| | | | | | 6.17 (MAE) | 3.66 (MAE) |
| [23] El Hajj [symbol] Kyriacou(2020) | MIMIC-II | [symbol] | 2-layer LSTM | [symbol] | 4.74 (SD) | [symbol] (SD) |
| | | | [symbol] | | 3.21 (MAE) | 1.59 (MAE) |
| [29] Schlesinger [symbol] | MIMIC-II | [symbol] | 8-layer CNN | Data Driven, | 8.65 (SD) | 4.48 (SD) |
| | | | [symbol] | [symbol] | 7.34 (MAE) | [symbol] (MAE) |
| [28] Slapnicar [symbol] | MIMIC-III | [symbol] | Comprehensive network | Data Driven, | | |
| | | | 15 ResNet blocks | [symbol] | [symbol] (MAE) | [symbol] (MAE) |
| | | | [symbol] blocks | | | |
| | | | [symbol] | | | |
| [25] [symbol] | Self prepared | [symbol] | [symbol] | [symbol] 19 | [symbol] | 0.31 ± [symbol] |
| | | | | features | (ME ± SD) | (ME ± SD) |
| [27] [symbol] | MIMIC-II | [symbol] | [symbol] | Data Driven | 0.19 ± [symbol] | [symbol] |
| | | | | PCA | (ME ± SD) | (ME ± SD) |
| [symbol] | MIMIC | [symbol] | ANN with [symbol] | [symbol] | [symbol] (SD) | 1. [symbol] (SD) |
| | | | hidden layer | | 4.02 (MAE) | [symbol] (MAE) |
| [symbol] | [symbol] | [symbol] | Regression [symbol] | [symbol] | −0.1 ± 6.5 | −6.6 ± 5.2 |
| | | | | | (ME ± SD) | (ME ± SD) |

*Instead of duration, the number of cardiac cycles were reported. For consistency in the table, the number is converted into duration, assuming 1 beat = 1 [symbol] . [symbol]
indicates data missing or illegible when filed

Three of the studies listed in the table, [23] (C. El Hajj and P. A. Kyriacou, “Cuffles and continuous blood pressures estimation from PPG signals using recurrent neural networks,” in Proc. of Annual International Conference of the IEEE Engineering in Medicine & Biology Society, 2020, pp. 4426-4272), [28](g. Slapnicar, N. Mlakar, and M. Lus?trek, “Blood pressure estimation from photoplethysmogram using a spectotemporal deep neural net-work,” Sensors, vol. 18, no. 15, p. 3420, 2019.) and [29] (O. Schlesinger, N. Vigderhouse, D. Eytan, and Y. Moshe, “Blood pressures estimation from PPG signals using convolutional neural networks and siamese network,” in Proc. of IEEE International Conference on Acoustics, Speech and Signal Processing, 2020, pp. 1135-1139.) utilize DNN architectures for feature learning. Comparing the results of the exemplary methods disclosed herein to the three studies, it is evident that the exemplary methods disclosed herein provides better MAE performance for SBP and DBP compared to [28] and [29], but lower performance compared to [23]. However, it is noted that [23] requires manual feature extraction, and much longer duration of PPG signal (7s) than the exemplary methods of the present disclosure. Furthermore, compared to all these three works, the exemplary methods of the present disclosure offers very fast training process, as it requires only a ridge regression process to determine all the required model parameters. Other DNN-based models listed in Table 3, in contrast, need to be trained from scratch.

Compared to studies in Table 3 that use manual feature engineering, the data-driven methods of the current disclosure offer simplicity in implementation, as VG image creation with single algorithm is much easier to implement compared to manual feature extraction, where multiple peak detection, feature calculation and validation algorithms have to be implemented to create the feature vector. In general, for methods relying on multiple manually-extracted features, as the number of required features increases, the risk of failure also increases. The increase in risk of failure is because, if even one of the required features cannot be extracted, the method fails to produce estimates of BP. The exemplary methods of the present disclosure, on the other hand, are robust in such circumstances, as any PPG signal presented to the algorithm is guaranteed to be converted to VG. When comparing the quantitative results, our data driven method, similar to other data-driven methods, shows better results compared to [25] (X. Xing, Z. Ma, M. Zhang, Y. Zhou, W. Dong, and M. Song, “An unobtrusive and calibration-free blood pressure estimation method using photoplethysmography and biometrics,” Scientific Reports, vol. 9, no. 1, p. 8611, 2019.) and slightly lower performance compared to [23], [26] (S. G. Khalid, J. Zhang, F. Chen, and D. Zheng, “Blood pressure esti-mation using photoplethysmography only: comparison between different machine learning approaches,” Journal of Healthcare Engineering, 2018, doi: 10.1155/2018/1548647.) and [53] (L. Wang, W. Zhou, Y. Xing, and X. Zhou, “A novel neural network model for blood pressure estimation using photoplethesmography with-out electrocardiogram,” Journal of Healthcare Engineering, 2018, doi: 10.1155/2018/7804243.). Compared to data-driven works, the methods of the present disclosure provide better or comparable BP estimation performance, while using shorter PPG duration compared to, and significantly smaller number of training parameters compared to all three.

### Example 4

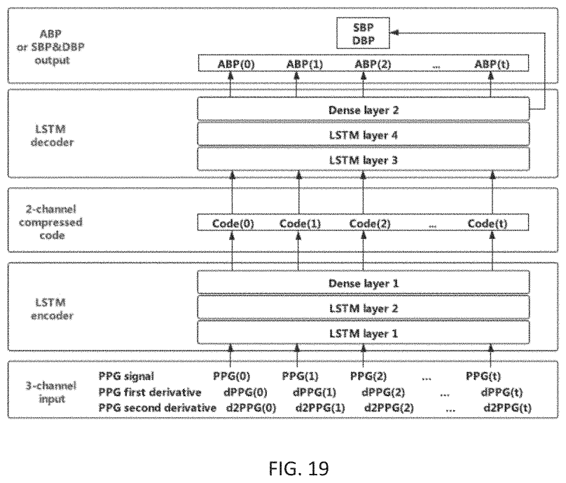

Long-short term memory (LSTM) is another type of recurrent neural network. To investigate how the methods of the present disclosure compare against using LSTM, the LSTM model proposed by L. N. Harfiya, C.-C. Chang, and Y.-H. Li, “Continuous blood pressure estimation using exclusively photopletysmography by LSTM-based signal-to-signal translation,” Sensors, vol. 21, no. 9:2952, pp. 1-15, 2021, Art. no. 2952, was implemented, as it shares similar properties with the present method in terms of requiring only PPG signal to estimate BP. The LSTM model is shown in FIG. 19. The LSTM model was implemented and tested using the data in Example 1 from 3-peak setting. The number of windows were slightly reduced to 45175 as the LSTM method requires precise alignment of PPG and ABP signals. 70%, 15% and 15% of the data was used for training, validation, and testing, respectively.

Two comparison studies were performed. First, labels of SBP and DBP were extracted from ABP for each window, to train and test the LSTM model. The estimation performance results are summarized in row B of Table 5, suggesting that the LSTM model fails to work in this case due to offering large estimation error and very low correlation coefficient. In the second study, the LSTM model was trained to estimate the ABP signal from the PPG input, instead of estimating SBP and DBP values directly. After training, estimates for SBP and DBP values were extracted by finding the highest and the lowest amplitudes of the estimated ABP for each PPG window. The results are summarized in row C of Table 5, indicating significantly improved performance compared to the first study. When comparing to the best case estimation results using the methods of the present disclosure (row A of Table 5), LSTM offers worse SBP estimation performance, and slightly better DBP estimation performance.

TABLE 5

| | Window | | SBF | | | | DBP | | | |
|---|---|---|---|---|---|---|---|---|---|---|
| Model | Setting | | R | ME ±SD | MAE | RMSE | R | ME ± SD | MAE | RMSE |
| A: This work, | 3-peak | (N = 57757) | 0.88 | −0.00 ± 8.46 | 6.17 | 8.46 | 0.84 | −0.04 ± 5.36 | 3.66 | 5.36 |
| using AlexNet | | | | | | | | | | |
| B: 4-layer LSTM | 3-peak | (N = 45175)* | 0.25 | 0.40 ± 17.4 | 13.86 | 17.40 | 0.04 | −0.65 ± 9.41 | 6.89 | 9.44 |
| with self-supervised | | | | | | | | | | |
| pre-training, trained | | | | | | | | | | |
| with SBP and DBP labels | | | | | | | | | | |
| C. 4-layer LSTM with | 3-peak | (N = 45175)* | 0.84 | 5.52 ± 10.01 | 8.35 | 11.43 | 0.85 | −0.90 ± 5.06 | 3.14 | 5.14 |
| self-supervised pre- | | | | | | | | | | |
| training, trained with | | | | | | | | | | |
| ABP signal | | | | | | | | | | |

*Due to the requirement of precise alignment of PPG and ABP signals, the number of windows were slightly reduced.

In summary, although the LSTM offered a good estimation performance from the second study described above, the results were obtained based on the condition of having access to continuous recordings of the reference BP (here the ABP signal) for training a good LSTM model. However, generally access to continuous recording of the reference BP signal (which would either require invasive methods or use of expensive instruments such as Finapres) is not possible or practical. On the contrary, our transfer-learning-based method is generalized and requires only SBP and DBP values for training the model, which can be easily retrieved via consumer-friendly cuff-based BP monitors.

In some embodiments, the exemplary methods of the present disclosure may be implemented into a wearable system. Furthermore, the closeness of BP-estimation accuracy results obtained from using feature vectors extracted from VG_{POS} and from VG_{INV} indicates potential redundancy and co-linearity. In some embodiments, pre-trained layers of the CNN may be used to determine the most significant features extracted from the VG images that has high correlation with BP values, which could be helpful for further reducing the dimension of the feature vectors and improving the efficiency of our algorithm.

As discussed above, the systems and methods disclosed herein achieve the goals of: 1) using only PPG signal (thereby, requiring minimal hardware and being user friendly), 2) eliminating the need of individual calibration and manual feature engineering, 3) enabling deep learning models to be applied with low computational budget on small-scale datasets, and 4) offering comparable BP-estimation performance. As such, the systems and method disclosed herein provide computationally-efficient cuff-less, continuous and patient-friendly BP monitoring.

At least some aspects of the present disclosure will now be described with reference to the following numbered clauses.

1. A method may include:

receiving, by at least one processor, a plurality of digital signal data from at least one sensor device of a plurality of sensor devices;

dynamically converting, by the at least one processor, the plurality of digital signal data into at least one visibility point in a plurality of visibility points based on at least one time series vector of a plurality of time series vectors associated with the plurality of digital signal data;

generating, by the at least one processor, utilizing the at least one visibility point, at least one image to preserve the at least one time series vector associated with the plurality of digital signal data, where the at least one time series vector is a shape within the at least one image;

extracting, by the at least one processor, at least one feature metric of a plurality of feature metrics from the at least one image based on an analysis of a pre-trained machine learning algorithm;

automatically determining, by the at least one processor, utilizing a transfer learning algorithm, a first position of at least one node in a plurality of nodes within the at least one image based on a relationship between the at least one feature metric and the at least one time series vector associated with the plurality of digital time series data; and

predicting, by the at least one processor, utilizing the transfer learning algorithm, a second position of the at least one node in the plurality of nodes based on the analysis of the pre-trained machine learning algorithm and the relation between the at least one feature metric and the at least one time series vector.

2. The method according to clause 1, where the plurality of digital signal data includes a plurality of physiological data signals.

3. The method according to clause 1 or 2, where the at least one visibility point preserves a plurality of temporal information associated with a data waveform based on the plurality of digital signal data.

4. The method according to clause 1, 2, or 3, where the pre-trained machine learning algorithm includes a deep conventional neural network.

5. The method according to clause 1, 2, 3, or 4, where predicting the second of the at least one node in the plurality of nodes includes estimating a systolic blood pressure and a diastolic blood pressure associated with at least one user in a plurality of users based on the plurality of feature metrics and the plural of time series vectors.

6. The method of clause 1, where the pre-trained learning algorithm is one of AlexNet, VGG-19 or Inception v3.

7. The method of clause 1, where the plurality of digital signal data includes PPG data and reference BP data.

8. The method of clause 1, where the at least one image is a visibility graph (VG).

9. The method of clause 1, further comprising applying a selection procedure to the plurality of digital signal data to remove at least one of:
duplicated digital signal data,
digital signal data of poor quality, or
digital signal with less than a predetermined number of systolic peaks.

10. The method of clause 1, further comprising classifying the feature metric into one of at least two classes.

11. A system including:

at least one processor configured to execute software instructions, where the software instructions, when executed, cause the at least one processor to perform steps to:
receive a plurality of digital signal data from at least one sensor device of a plurality of sensor devices;
dynamically convert the plurality of digital signal data into at least one visibility point in a plurality of visibility points based on at least one time series vector of a plurality of time series vectors associated with the plurality of digital signal data;
generate, utilizing the at least one visibility point, at least one image to preserve the at least one time series vector associated with the plurality of digital signal data, where the at least one time series vector is a shape within the at least one image;
extract at least one feature metric of a plurality of feature metrics from the at least one image based on an analysis of a pre-trained machine learning algorithm;
automatically determine, utilizing a transfer learning algorithm, a first position of at least one node in a plurality of nodes within the at least one image based on a relationship between the at least one feature metric and the at least one time series vector associated with the plurality of digital time series data; and
predict, utilizing the transfer learning algorithm, a second position of the at least one node in the plurality of nodes based on the analysis of the pre-trained machine learning algorithm and the relation between the at least one feature metric and the at least one time series vector.

12. The system of clause 11, where the plurality of digital signal data includes a plurality of physiological data signals.

13. The system of clause 11, where the at least one visibility point preserves a plurality of temporal information associated with a data waveform based on the plurality of digital signal data.

14. The system of clause 11, where the pre-trained machine learning algorithm includes a deep conventional neural network.

15. The system of clause 11, where predicting the second of the at least one node in the plurality of nodes includes estimating a systolic blood pressure and a diastolic blood pressure associated with at least one user in a plurality of users based on the plurality of feature metrics and the plural of time series vectors.

16. The system of clause 11, where the pre-trained learning algorithm is one of AlexNet, VGG-19 or Inception v3.

17. The system clause 11, where the plurality of digital signal data includes PPG data and reference BP data.

18. The system of clause 11, where the at least one image is a visibility graph (VG).

19. The system of clause 11, where the software instructions, when executed, further cause the at least one processor to perform steps to:

apply a selection procedure to the plurality of digital signal data to remove at least one of:
duplicated digital signal data,
digital signal data of poor quality, or
digital signal with less than a predetermined number of systolic peaks.

The system of clause 11, where the software instructions, when executed, further cause the at least one processor to perform steps to classify the feature metric into one of at least two classes.

While one or more embodiments of the present disclosure have been described, it is understood that these embodiments are illustrative only, and not restrictive, and that many modifications may become apparent to those of ordinary skill in the art, including that various embodiments of the inventive methodologies, the illustrative systems and platforms, and the illustrative devices described herein can be utilized in any combination with each other. Further still, the various steps may be carried out in any desired order (and any desired steps may be added and/or any desired steps may be eliminated).

What is claimed is:

1. A computer-implemented method comprising:
receiving, by at least one processor, a plurality of digital signal data from at least one sensor device of a plurality of sensor devices;
dynamically converting, by the at least one processor, the plurality of digital signal data into at least one visibility point in a plurality of visibility points based on at least one time series vector of a plurality of time series vectors associated with the plurality of digital signal data;
generating, by the at least one processor, utilizing the at least one visibility point, at least one image to preserve the at least one time series vector associated with the plurality of digital signal data, wherein the at least one time series vector is a shape within the at least one image;
extracting, by the at least one processor, at least one feature metric of a plurality of feature metrics from the at least one image based on an analysis of a pre-trained machine learning algorithm;
automatically determining, by the at least one processor, utilizing a transfer learning algorithm, a first position of at least one node in a plurality of nodes within the at least one image based on a relationship between the at least one feature metric and the at least one time series vector associated with the plurality of digital time series data; and
predicting, by the at least one processor, utilizing the transfer learning algorithm, a second position of the at least one node in the plurality of nodes based on the analysis of the pre-trained machine learning algorithm and the relation between the at least one feature metric and the at least one time series vector.

2. The computer-implemented method of claim 1, wherein the plurality of digital signal data comprises a plurality of physiological data signals.

3. The computer-implemented method of claim 1, wherein the at least one visibility point preserves a plurality of temporal information associated with a data waveform based on the plurality of digital signal data.

4. The computer-implemented method of claim 1, wherein the pre-trained machine learning algorithm comprises a deep conventional neural network.

5. The computer-implemented method of claim 1, wherein predicting the second of the at least one node in the plurality of nodes comprises estimating a systolic blood pressure and a diastolic blood pressure associated with at least one user in a plurality of users based on the plurality of feature metrics and the plural of time series vectors.

6. The computer-implemented method of claim 1, wherein the pre-trained learning algorithm is one of AlexNet, VGG-19 or Inception v3.

7. The computer-implemented method of claim 1, wherein the plurality of digital signal data comprises PPG data and reference BP data.

8. The computer-implemented method of claim 1, wherein the at least one image is a visibility graph (VG).

9. The computer-implemented method of claim 1, further comprising applying a selection procedure to the plurality of digital signal data to remove at least one of:
duplicated digital signal data,
digital signal data of poor quality, or
digital signal with less than a predetermined number of systolic peaks.

10. The computer-implemented method of claim 1, further comprising classifying the feature metric into one of at least two classes.

11. A system comprising:
at least one processor configured to execute software instructions, wherein the software instructions, when executed, cause the at least one processor to perform steps to:
receive a plurality of digital signal data from at least one sensor device of a plurality of sensor devices;
dynamically convert the plurality of digital signal data into at least one visibility point in a plurality of visibility points based on at least one time series vector of a plurality of time series vectors associated with the plurality of digital signal data;
generate, utilizing the at least one visibility point, at least one image to preserve the at least one time series vector associated with the plurality of digital signal data, wherein the at least one time series vector is a shape within the at least one image;
extract at least one feature metric of a plurality of feature metrics from the at least one image based on an analysis of a pre-trained machine learning algorithm;
automatically determine, utilizing a transfer learning algorithm, a first position of at least one node in a plurality of nodes within the at least one image based on a relationship between the at least one feature metric and the at least one time series vector associated with the plurality of digital time series data; and
predict, utilizing the transfer learning algorithm, a second position of the at least one node in the plurality of nodes based on the analysis of the pre-trained machine learning algorithm and the relation between the at least one feature metric and the at least one time series vector.

12. The system of claim 11, wherein the plurality of digital signal data comprises a plurality of physiological data signals.

13. The system of claim 11, wherein the at least one visibility point preserves a plurality of temporal information associated with a data waveform based on the plurality of digital signal data.

14. The system of claim 11, wherein the pre-trained machine learning algorithm comprises a deep conventional neural network.

15. The system of claim 11, wherein predicting the second of the at least one node in the plurality of nodes comprises estimating a systolic blood pressure and a diastolic blood pressure associated with at least one user in a plurality of users based on the plurality of feature metrics and the plural of time series vectors.

16. The system of claim 11, wherein the pre-trained learning algorithm is one of AlexNet, VGG-19 or Inception v3.

17. The system claim 11, wherein the plurality of digital signal data comprises PPG data and reference BP data.

18. The system of claim 11, wherein the at least one image is a visibility graph (VG).

19. The system of claim 11, wherein the software instructions, when executed, further cause the at least one processor to perform steps to:
apply a selection procedure to the plurality of digital signal data to remove at least one of:
duplicated digital signal data,
digital signal data of poor quality, or
digital signal with less than a predetermined number of systolic peaks.

20. The system of claim 11, wherein the software instructions, when executed, further cause the at least one processor to perform steps to classify the feature metric into one of at least two classes.
